(19) United States
(12) Patent Application Publication
(10) Pub. No.: US 2006/0236430 A1
(43) Pub. Date: Oct. 19, 2006

(54) Methods and compositions for producing male sterile plants

(76) Inventors: Helmut Maucher, Halle/Saale (DE); Otto Miersch, Halle/Saale (DE); Claus Wasternack, Halle/Saale (DE); Luc Varin, Montreal (CA)

Correspondence Address: Choate, Hall & Stewart, Two International Place, Boston, MA, 02110, US

(21) Appl. No.: 10/542,113

(22) PCT Filed: Jan. 13, 2004
(86) PCT No.: PCT/CA04/00006

§ 371 Date: Feb. 24, 2006

(30) Foreign Application Priority Data
Jan. 13, 2003 (CA) 2,414,487

(51) Int. Cl. A01H 1/00; C12N 15/82; A01N 37/00; C12N 5/04

(52) U.S. Cl. 800/287; 435/468; 435/419; 504/307

(57) ABSTRACT

The invention relates to methods of producing male sterile plants by increasing the level of activity of the hydroxyjasmonate sulfotransferase by genetic modification of such plant. Furthermore, the present invention provides methods to restore the fertility of the male sterile phenotype observed in such genetically modified plants by the application of a composition containing at least one jasmonate and an acceptable carrier onto the male sterile flowers.

### FIELD OF THE INVENTION

The present invention relates to methods to produce male sterile plants by decreasing the level of 11-and/or 12-hydroxyjasmonate via increasing the endogenous activity of a 11-/12-hydroxyjasmonic acid sulfotransferase and/or decreasing the activity of a jasmonic acid 11-/12-hydroxylase, and to restore the male sterile phenotype in said plants by applying to said plants a composition containing a jasmonate.

### BRIEF DESCRIPTION OF THE PRIOR ART

There are several direct and indirect evidences for the involvement of jasmonic acid (JA) and related cyclopentanones, collectively called jasmonates in flower development (Wastemack and Hause (2002), Prog Nucleic Acid Res Mol Biol 72:165-221). For instance, Arabidopsis thaliana mutants in JA biosynthesis or signaling are male sterile as a consequence of improper timing of anther and pollen development (Feys et al. (1994) Plant Cell 6: 751-759). A role in female reproductive development has also been attributed to jasmonates. For instance, the tomato jai-1 mutant which is insensitive to the exogenous application of methyl jasmonate (MeJA) and cannot express defense-related genes in response to wounding was found to be female sterile (Li et al. (2001) Plant Physiol. 127:1414-1417). The species-specific difference in the requirement of jasmonates for the development of male or female gametophyte has yet to be explained. Finally, relatively high levels of jasmonates are found in the developing reproductive organs as compared to leaves and specific jasmonates such as JA tyramine conjugate and 12-hydroxyjasmonate (12-OHJA) are present in flowers (Miersch et al. (1998) Phytochemistry 47: 327-329).

The inventors have demonstrated that 11- and/or 12-OHJA (FIG. 1) are required for proper anther development. First, they demonstrated that the exogenous application of 12-OHJA to the inflorescence of the A. thaliana opr3 mutant deficient in JA biosynthesis restored the male sterile phenotype. Furthermore, they demonstrated that the overexpression of the A. thaliana AtST2a gene encoding a 11-/12-OHJA sulfotransferase in transgenic tobacco led to a male sterile phenotype that could be restored by the exogenous application of JA or 12-OHJA.

Many functions have been associated with jasmonate metabolites such as 12-hydroxyjasmonic acid and/or 11-hydroxyjasmonic acid. For instance, U.S. Pat. No. 5,935,809, suggests the use of jasmonate for inducing plant defense mechanisms. U.S. Pat. No. 5,814,581 describes a plant growth promoter composition comprising jasmonate and brassinolide as active ingredients and Tazaki (Japanese kokai 292220 (A) published Apr. 3 1990, and patent application no 63-242432, filed Sep. 29, 1988); Yoshihara et al. (1989), Agric. Biol. Chem. 53: 2835-2837, Matsuki et al. (1992), Biosci. Biotech. Biochem. 56: 1329.; and Koda and Okazawa (1988), Plant Cell Physiol. 29: 969), suggest the use of 12-hydroxyjasmonic acid for inducing tuber formation in potatoes. None of these documents disclose nor suggest that it is possible to produce male sterile plants by increasing in-vivo sulfonation of hydroxyjasmonates or by decreasing the synthesis of 11- and/or 12-OHJA.

Accordingly, there is a need for effective methods to produce male sterile plants that can be applied to all flowering plants and for methods to restore the male sterile phenotype. There is also a need for plants genetically modified to be male sterile.

### SUMMARY OF THE INVENTION

An object of the present invention is to provide a method that satisfies the above mentioned needs.

Accordingly, the present invention provides for a method of producing a male sterile plant characterized in that it comprises the step of decreasing the level of 11-and/or 12-hydroxyjasmonate by increasing in said plant the level of in-vivo sulfonation of hydroxyjasmonates or decreasing the level of synthesis of 11-and/or 12-hydroxyjasmonate.

Another object of the present invention is to provide a plant cell transformation vector capable of facilitating transfer and expression of an exogenous nucleic acid into an isolated cell and/or facilitating integration of an exogenous nucleic acid into genome of the cell, characterized in that the vector comprises at least one promoter sequence, one enhancer sequence and one exogenous nucleic acid sequence. The promoter is a constitutive expression promoter or an inducible promoter. The exogenous nucleic acid is selected from the group consisting of nucleic acid sequence having at least 50% homology with SEQ ID no. 1 or SEQ ID no. 2 and a nucleic acid encoding for an amino acid sequence having at least 50% homology with amino acid sequence of SEQ ID no. 3 or SEQ ID no. 4.

Another object of the present invention is to provide a method for producing a male sterile plant which comprises:
introducing into a cell of a suitable plant an exogenous nucleic acid molecule via the vector as defined above;
regenerating a transgenic plant from the cell; and where necessary
growing the transgenic plant for a time and under conditions sufficient to permit expression of the exogenous nucleic acid sequence and thereby stimulating expression of the hydroxyjasmonic acid sulfotransferase.

Another object of the present invention is to provide a genetically modified male sterile plant, characterized in that its endogenous level of 11- or 12-hydroxyjasmonate is lower than the endogenous level of 11- or 12-hydroxyjasmonate in a non genetically modified plant.

A further object of the present invention is to provide a genetically modified plant characterized in that its endogenous level of 11- or 12- hydroxyjasmonic acid sulfotransferase is higher than the endogenous level of 11- or 12-hydroxyjasmonic acid sulfotransferase in a non genetically modified plant.

According to another object of the invention, there is provided a composition for restoring normal anther development in a genetically modified male sterile plant according to the present invention, comprising at least one jasmonate and an acceptable carrier.

According to another object of the present invention, there is provided a composition for restoring normal anther development in a genetically modified male sterile plant, comprising at least one 11-/12-OHJA sulfotransferase inhibitor and an acceptable carrier.

Yet, according to another object of the invention, there is provided methods for restoring normal anther development in the genetically modified male sterile plant, comprising the step of applying on male sterile flowers of said plant one of the compositions as defined above.

### BRIEF DESCRIPTION OF THE DRAWINGS

FIG. 1 shows the chemical structures of 11-hydroxyjasmonic acid and 12-hydroxyjasmonic acid.

FIG. 2 shows the results of a Northern blot experiment with mRNA extracted from selected transgenic lines as compared to wild type.

FIG. 3 is a composite picture showing the phenotype of flowers from transgenic Nicotiana tabacum plants expressing the AtST2a gene under the control of a constitutive promoter (CaMV35S) compared to flowers from wild type non-transgenic plants (WT).

FIG. 4 shows the results of the quantification of jasmonates in dissected tissues from wild type and mutant flowers.

FIG. 5 shows the results of a normalization experiment. Treatments of the apex of transgenic plants for 14 days with 12-OHJA led to the restoration of the mutant phenotype and to the production of viable pollen.

FIG. 6 shows nucleotide sequence of AtST2a gene (SEQ ID NO 1) taken from the GenBank® database (accession number NM_{—}120783).

FIG. 7 shows the deduced amino acid sequence (SEQ ID NO 3) of the protein encoded by the AtST2a gene shown in FIG. 6.

FIG. 8 shows the nucleotide sequence of AtST2b gene (SEQ ID NO 2) taken from the GenBank® database (accession number NM_{—}120782).

FIG. 9 shows the deduced amino acid sequence (SEQ ID NO 4) of the protein encoded by the AtST2b gene shown in FIG. 8.

### DETAILED DESCRIPTION OF THE INVENTION

A) Definitions

In order to provide an even clearer and more consistent understanding of the specification, including the scope given herein to such terms, the following definitions are provided:

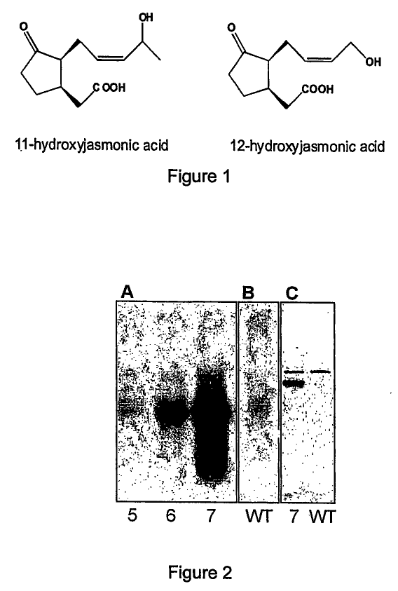

11-hydroxyjasmonic acid: 3-Oxo-2-(4-hydroxy-2-pentenyl)-cyclopentane-1-acetic acid. Its chemical structure is shown in FIG. 1.

11-hydroxyjasmonic acid sulfate: 3-Oxo-2-(4-hydroxysulfonyloxy-2-pentenyl)-cyclopentane-1-acetic acid

12-hydroxyjasmonic acid: 3-Oxo-2-(5-hydroxy-2-pentenyl)-cyclopentane-1-acetic acid. Its chemical structure is shown in FIG. 1.

12-hydroxyjasmonic acid sulfate: 3-Oxo-2-(5-hydroxysulfonyloxy-2-pentenyl)-cyclopentane-1-acetic acid.

Antisense: Refers to nucleic acids molecules capable of regulating the expression of a corresponding gene in a plant. An antisense molecule as used herein may also encompass a gene construct comprising a structural genomic gene, a cDNA gene or part thereof in reverse orientation relative to its or another promoter. Typically antisense nucleic acid sequences are not templates for protein synthesis but yet interact with complementary sequences in other molecules (such as a gene or RNA) thereby causing the function of those molecules to be affected.

Exogenous nucleic acid: A nucleic acid sequence (such as cDNA, cDNA fragments, genomic DNA fragments, antisense RNA, oligonucleotide) which is not normally part of a plant genome. The “exogenous nucleic acid” may be from any organism or purely synthetic. Typically, the “exogenous nucleic acid sequence” encodes a plant gene such as AtST2a, AtST2b or functional homologues of these genes.

Expression: The process whereby an exogenous nucleic acid, such as a nucleic acid sequence encoding a gene, is transcribed into a mRNA and afterwards translated into a peptide or a protein, in order to carry out its function, if any.

Functional homologue: Refers to a molecule having at least 50%, more preferably at least 55%, even more preferably at least 60%, still more preferably at least 65-70%, and yet even more preferably greater than 85% similarity at the level of nucleotide or amino acid sequence to at least one or more regions of a given nucleotide or amino acid sequence. According to preferred embodiments of the present invention, the terms “functional homologue” refer to proteins or nucleic acid sequences encoding an enzyme having a substantially similar biological activity as 11- or 12-hydroxyjasmonate sulfotransferase and isoenzyme thereof. Such a functional homologue may exist naturally or may be obtained following a single or multiple amino acid substitutions, deletions and/or additions relative to the naturally occurring enzyme using methods and principles well known in the art. A functional homologue of a protein may or may not contain post-translational modifications such as covalently linked carbohydrate, if such modification is not necessary for the performance of a specific function. It should be noted, however, that nucleotide or amino acid sequences may have similarities below the above given percentages and still encode a 11- or 12-hydroxyjasmonate sulfotransferase-like molecule, and such molecules may still be considered within the scope of the present invention where they have regions of sequence conservation.

Geneticinucleotide sequence: These terms are used herein in their most general sense and encompass any contiguous series of nucleotide bases encoding directly, or via a complementary series of bases, a sequence of amino acids comprising a hydroxyjasmonic acid sulfotransferase molecule, and more particularly a 11- or 12-OHJA sulfotransferase. Such a sequence of amino acids may constitute a full-length 11- or 12-OHJA sulfotransferase such as is set forth in SEQ ID No:1 and SEQ ID No:2 or an active truncated form thereof or a functional mutant, derivative, part, fragment, homologue or analogue thereof, or may correspond to a particular region such as an N-terminal, C-terminal or internal portion of the enzyme.

Genetic modification or genetic engineering: Refers to the introduction of an exogenous nucleic acid into one or more plant cells to create a genetically modified plant. Methods for genetically modifying a plant are well known in the art. In some cases, in may be preferable that the genetic modification is permanent such that the genetically modified plant may regenerate into whole, sexually competent, viable genetically modified plants. A plant genetically modified in a permanent manner would preferably be capable of self-pollination or cross-pollination with other plants of the same species, so that the exogenous nucleic acid, carried in the germ line, may be inserted into or bred into agriculturally useful plant varieties.

Endogenous level(s): Refers to the amount of a given substance which is normally found in a plant (intrinsic) at a given time and stage of growth. Reference herein is made to the altering of the endogenous level of a compound or of an enzyme activity relating to an elevation or reduction in the compound's level or enzyme activity of up to 30% or more preferably of 30-50%, or even more preferably 50-75% or still more preferably 75% or greater above or below the normal endogenous or existing levels. The levels of a compound or the levels of activity of an enzyme can be assayed using known method and techniques.

Inhibitor: As used herein, the terms “inhibit”, “inhibition”, “inhibitory”, and “inhibitor” all refer to a function of substantially reducing a biological activity or function. Such reduction in activity or function can, for example, be in connection with a cellular component (e.g., an enzyme), or in connection with a cellular process (e.g., synthesis of a particular protein). With respect to an “inhibitor of an enzyme”, such an inhibitor may bind to or change the conformation of the active site of an enzyme, in a reversible or irreversible way. More specifically, the term “inhibitor” extends to compounds that interact with the enzyme 11- or 12-OHJA sulfotransferase.

Isolated nucleic acid molecule: Means a genetic sequence in a non-naturally-occurring condition. Generally, this means isolated away from its natural state or formed by procedures not necessarily encountered in its natural environment. More specifically, it includes nucleic acid molecules formed or maintained in vitro, including genomic DNA fragments, recombinant or synthetic molecules and nucleic acids in combination with heterologous nucleic acids such as heterologous nucleic acids fused or operably-linked to the genetic sequences of the present invention. The term “isolated nucleic acid molecule” also extends to the genomic DNA or cDNA or part thereof, encoding a hydroxyjasmonic acid sulfotransferase, preferably a 11- or 12-OHJA sulfotransferase, or a functional mutant, derivative, part, fragment, homologue or analogue of 11- or 12-OHJA sulfotransferase in reverse orientation relative to its or another promoter. It further extends to naturally occurring sequences following at least a partial purification relative to other nucleic acid sequences. The term isolated nucleic acid molecule as used herein is understood to have the same meaning as nucleic acid isolate.

Plant: refers to a whole plant or a part of a plant comprising, for example, a cell of a plant, a tissue of a plant, an explant, or seeds of a plant. This term further contemplates a plant in the form of a suspension culture or a tissue culture including, but not limited to, a culture of calli, protoplasts, embryos, organs, organelles, etc.

Similarity/Complementarity: In the context of nucleic acid sequences, these terms mean a hybridizable similarity under low, alternatively and preferably medium and alternatively and most preferably high stringency conditions, as defined below. Such a nucleic acid is useful, for example, in screening hydroxyjasmonic acid sulfotransferase genetic sequences, preferably a 11- or 12-hydroxyjasmonic acid sulfotransferase genetic sequences from various sources or for monitoring an introduced genetic sequence in a transgenic plant. The preferred oligonucleotide is directed to a conserved hydroxyjasmonic acid sulfotransferase, preferably a 11- or 12-hydroxyjasmonic acid sulfotransferase genetic sequence or a sequence conserved within a plant genus, plant species and/or plant cultivar or variety.

Stringency: For the purpose of defining the level of stringency, reference can conveniently be made to Maniatis et al. (1982) at pages 387-389, and especially paragraph 11. A low stringency is defined herein as being in 4-6×SSC/1% (w/v) SDS at 3745° C. for 2-3 hours. Depending on the source and concentration of nucleic acid involved in the hybridization, alternative conditions of stringency may be employed such as medium stringent conditions which are considered herein to be 1-4×SSC/0.5-1% (w/v) SDS at greater than or equal to 45° C. for 2-3 hours or high stringent conditions considered herein to be 0.1-1×SSC/0.1-1.0% SDS at greater than or equal to 60° C. for 1-3 hours.

Transformed plant: Refers to introduction of an exogenous nucleic acid, typically a gene, into a whole plant or a part thereof, and expression of the exogenous nucleic acid in the plant.

Transgenic plant: Refers to a whole plant or a part thereof stably transformed with an exogenous nucleic acid introduced into the genome of an individual plant cell using genetic engineering methods.

Vector: A self-replicating RNA or DNA molecule, which can be used to transfer an RNA or DNA segment from one organism to another. Vectors are particularly useful for manipulating genetic constructs and different vectors may have properties particularly appropriate to express protein(s) in a recipient during cloning procedures and may comprise different selectable markers. Bacterial plasmids are commonly used vectors. Preferably, the vectors of the invention are capable of facilitating transfer of a nucleic acid into a plant cell and/or facilitating integration into a plant genome.

B) General Overview of the Invention

The present inventors have now discovered that it is possible to produce male sterile plants by decreasing the level of 11-and/or 12- hydroxyjasmonate via increasing in the plant in-vivo sulfonation of hydroxyjasmonates or decreasing synthesis of 11-and/or 12- hydroxyjasmonate. Therefore, the present invention is directed to a genetically modified male sterile plant as well as a plant cell transformation vector. The present invention is also concerned with methods for producing such plants or for restoring normal anther development in such produced male sterile plant.

As mentioned above, the present invention provides a genetically modified male sterile plant. More specifically, the genetically modified male sterile plant of the invention is characterized in that its endogenous level of 11- or 12-hydroxyjasmonate is lower than the endogenous level of 11- or 12-hydroxyjasmonate in a non genetically modified plant. The genetically modified male sterile plant of the invention is preferably further characterized in that its endogenous level of 11- or 12- hydroxyjasmonic acid sulfotransferase is higher than the endogenous level of 11- or 12- hydroxyjasmonic acid sulfotransferase in a non genetically modified plant.

As used herein, the expression “higher than” refers to a substantial increase of the level of the sulfotransferase in the genetically modified plant relative to its level in the non-genetically modified plant. Such a substantial increase is preferably at least 25% in the level of the sulfotransferase in the genetically modified plant relative to its level in the non-genetically modified plant.

As used herein, the expression “lower than” refers to a substantial reduction in the level of 11- or 12- hydroxyjasmonate in the genetically modified plant relative to its level in a non-genetically modified plant. Such a substantial reduction is preferably at least 25% OK in the level of 11- or 12-hydroxyjasmonate in the genetically modified plant relative to its level in a non-genetically modified plant.

It will be understood that in accordance with the present invention, that having a higher endogenous level of hydroxyjasmonic acid sulfotransferase in a genetically modified male sterile plant of the invention will allow the sulfonation of 12-hydroxyjasmonic acid and/or 11-hydroxyjasmonic acid. Consequently, the resulting genetically modified male sterile plant will advantageously have a decreased endogenous level of at least one given compound of the jasmonate family selected from the group consisting of 12-OHJA, glucoside of 12-OHJA, 12-hydroxymethyljasmonic acid, glucoside of 12-hydroxymethyljasmonic acid, 11-OHJA, glucoside of 11-OHJA, 11-hydroxymethyljasmonic acid, and glucoside of 11-hydroxymethyljasmonic acid as well as the amino acid conjugates of 12-OHJA, glucoside of 12-OHJA, 11-OHJA, glucoside of 11-OHJA, when compared to the corresponding non-genetically modified plant.

According to another embodiment, a genetically modified male sterile plant of the invention is advantageously produced by a method that comprises the step of decreasing the level of 11-and/or 12- hydroxyjasmonate by increasing in said plant the level of in-vivo sulfonation of hydroxyjasmonates or decreasing the level of synthesis of 11-and/or 12- hydroxyjasmonate. According to a preferred embodiment, the level of in-vivo sulfonation of hydroxyjasmonates is increased by increasing in said plant the endogenous activity of a hydroxyjasmonate sulfotransferase, whereas the level of synthesis of 11-and/or 12-hydroxyjasmonate is decreased by decreasing in said plant the activity of a jasmonic acid 11-/ 12- hydroxylase.

As can be appreciated, the increasing of the level of in-vivo sulfonation of hydroxyjasmonates or the decreasing of the level of synthesis of 11-and/or 12-hydroxyjasmonate is achieved by any process known to one skilled in the art, such as, but not limited to, a process selected from the group consisting of genetic modification of said plant, radiation mutagenesis of said plant, chemical mutagenesis of said plant and selection of natural mutants. More preferably, the process consists of genetic modification.

It will be understood that, in a situation where the decreasing of the level of synthesis of 11-and/or 12- hydroxyjasmonate is preferred, the decrease of the expression of the hydroxylase(s) responsible for the conversion of jasmonic acid to 11- and/or 12-OHJA can be achieved by a process such as antisense technology, by knockout of the gene, by expression of an antibody inhibiting the hydroxylase activity or by the expression of a ribozyme specific for the mRNA of the jasmonic acid to 11- and/or 12-hydroxylase.

In a preferred embodiment of the invention, the plant is modified by stimulating the expression of at least one gene selected from the group consisting of AtST2a, AtST2b and functional homologues thereof of at least one gene selected from: the group consisting of AtST2a, AtST2b, functional homologues thereof having at least 50% homology with SEQ ID no. 1 or SEQ ID no. 2 and a nucleic acid encoding for an amino acid sequence having at least 50% homology with amino acid sequence of SEQ ID no. 3 or SEQ ID no. 4.

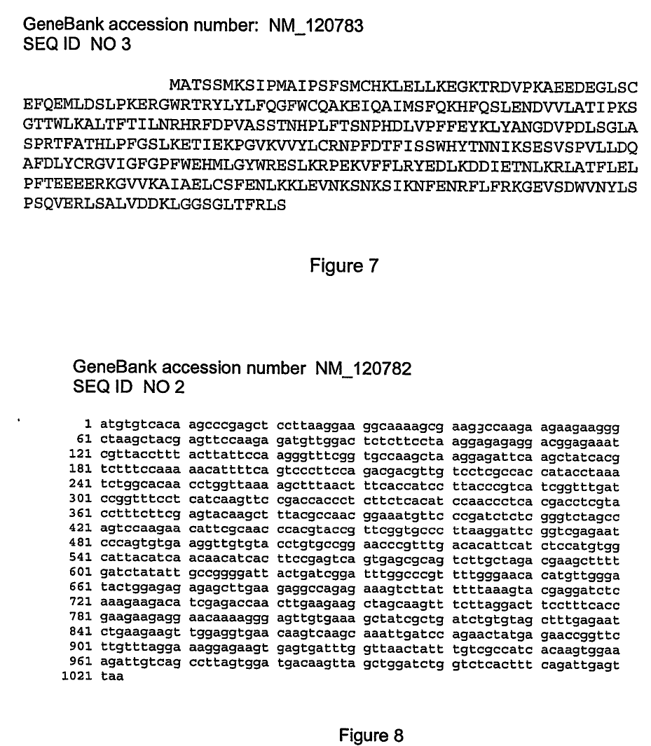

SEQ ID NO 1 (FIG. 6; GenBank®: accession number NM_{—}120783) corresponds to the gene AtST2a in Arabidopsis thaliana. SEQ ID NO 3 (FIG. 7) is an amino acid sequence deduced from SEQ ID NO 1. This amino acid sequence is of public domain and can be retrieved from GenBank®, accession number NM_{—}120783. The AtST2a gene from Arabidopsis thaliana encodes a sulfotransferase that sulfonates 12-OHJA and 11-OHJA with high specificity. This hydroxyjasmonic acid sulfotransferase exhibits high affinity for its substrate with a Km value of 11 μM for 12-OHJA and 60 μM for 11-OHJA. The enzyme did not accept structurally related compounds such as cucurbic acid, arachidonyl alcohol or prostaglandins. Maximum enzyme activity was observed at pH 7.5 in Tris/HCl buffer and did not require divalent cations for activity.

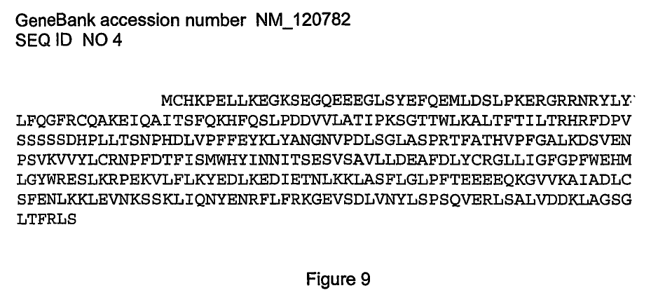

SEQ ID NO 2 (FIG. 8; GenBank®: accession number NM_{—}120782) corresponds to the gene AtST2b in Arabidopsis thaliana. SEQ ID NO 4 (FIG. 9) is an amino acid sequence deduced from SEQ ID NO 1. This amino acid sequence is of public domain and can be retrieved from GenBank®, accession number NM_{—}120782. Amino acid sequence alignment between SEQ ID NOS 3 and 4 indicates that they share 85% amino acid sequence identity and 92% similarity, suggesting that AtST2a and AtST2b encode isoenzymes.

The nucleic acid molecules contemplated herein may exist alone or in combination with a plant transformation vector. Consequently, another aspect of the invention is to provide a plant cell transformation vector capable of facilitating transfer and expression of an exogenous nucleic acid into an isolated cell and/or facilitating integration of an exogenous nucleic acid into genome of said cell, characterized in that said vector comprises at least one promoter sequence, one enhancer sequence such as an AMV translational enhancer, and one exogenous nucleic acid sequence. Preferably, the promoter sequence is a constitutive expression promoter such as an ubiquitin promoter, or an inducible promoter such as an ethanol-inducible promoter or a glucocorticoid-inducible promoter. More preferably, the promoter is CaMV 35S. The exogenous nucleic acid is selected from the group consisting of nucleic acid sequence having at least 50% homology with SEQ ID no. 1 or SEQ ID no. 2 and a nucleic acid encoding for an amino acid sequence having at least 50% homology with amino acid sequence of SEQ ID no. 3 or SEQ ID no. 4. Such a vector may, for example, be adapted for use in electroporation, microprojectile bombardment, Agrobacterium-mediated transfer or insertion via DNA or RNA viruses. The vector and/or the nucleic acid molecule contained therein may or may not need to be stably integrated into the plant genome. The vector may also replicate and/or express in prokaryotic cells. Preferably, the vector molecules or parts thereof are capable of integration into the plant genome.

The present invention is exemplified using nucleic acid sequences derived from Arabidopsis thaliana since this plant is commonly studied in and it represents a convenient and easily accessible source of material. However, one skilled in the art will immediately appreciate that similar sequences can be isolated from any number of sources such as other plants or certain microorganisms (e.g. fungi or bacteria). All such nucleic acid sequences encoding directly or indirectly a hydroxyjasmonic acid sulfotransferase are encompassed by the present invention regardless of their source. Examples of other suitable sources of genes encoding hydroxyjasmonic acid sulfotransferase include, but are not limited to Brassica napus, Solanum tuberosum, Solanum demissum, Nicotiana tabacum, Helianthus tuberosus and Astragalus complanatus

According to another embodiment, the present invention provides a method comprising the steps of:
a) introducing into a cell of a suitable plant an exogenous nucleic acid molecule via a vector according to the present invention described herein above, comprising a sequence of nucleotides encoding a plant hydroxyjasmonic acid sulfotransferase, preferably a 11- or 12-hydroxyjasmonic acid sulfotransferase;
b) regenerating a transgenic plant from the cell; and where necessary
c) growing the transgenic plant for a time and under conditions sufficient to permit expression of the exogenous nucleic acid sequence and thereby stimulating the expression of the plant hydroxyjasmonic acid sulfotransferase, preferably a 11- or 12-hydroxyjasmonic acid sulfotransferase.

The details of the construction of transgenic plants are known to those skilled in the art of plant genetic engineering and do not differ in kind from those practices which have previously been demonstrated to be effective in tobacco, petunia and other model plant species (e.g. electroporation, microprojectile bombardment, Agrobacterium-mediated transfer or insertion via DNA or RNA viruses). One skilled in the art will immediately recognize the variations applicable to the methods of the present invention, such as increasing the expression of the sulfotransferase naturally present in a target plant leading to the production of male sterile plants. The present invention, therefore, extends to all transgenic plants containing all or part of the nucleic acid sequence of the present invention, and/or any homologues or related forms thereof and in particular those transgenic plants which exhibit a male sterile phenotype.

In accordance with the present invention, the male sterile phenotype of the plants overexpressing the hydroxyjasmonate sulfotransferase can be advantageously restored. Therefore, another embodiment of the present invention is to provide a method for restoring normal anther development in the genetically modified male sterile plant of the invention by applying on male sterile flowers of the plant a composition as defined herein after. In a preferred embodiment, the step of applying said composition on male sterile flowers of said plant is achieved by soaking male sterile flowers in said composition for two minutes daily starting 7 days before appearance of a first flower bud and ending 7 days after appearance of a first flower.

The composition contemplated by the present invention comprises at least one jasmonate and an acceptable carrier. Preferably, the jasmonate is selected from the group consisting of: 11-hydroxyjasmonic acid, 12-hydroxyjasmonic acid, glucoside of 11-hydroxyjasmonic acid, glucoside of 12-hydroxyjasmonic acid, 11-hydroxymethyljasmonic acid, glucoside of 11-hydroxymethyljasmonic acid, 12-hydroxymethyljasmonic acid and glucoside of 12-hydroxymethyljasmonic acid. Structural analogs of the above mentioned compounds could also be used to restore the phenotype. In a preferred embodiment of the invention, the normal anther development in the genetically modified male sterile plant is restored by applying on the male sterile flowers of the plant a composition containing 100 μM of 12-hydroxyjasmonic acid or 50 μM methyl-jasmonic acid and an acceptable carrier. The composition preferably further contains TWEEN20™ for instance in the concentration of 0.05 weight percent of its total weight.

As used herein, the expression “an acceptable carrier” means a vehicle for containing a jasmonate that can be introduced in a plant without adverse effects. Suitable carriers known to one skilled in the art include, but are not limited to, a solvent such as water, oil or alcohol. The carrier may include auxiliary agents such as fertilizers and growth regulators. The composition contemplated by the present invention may also be formulated with emulsifying agents in the presence or absence of fungicides or insecticides, if required. The precise amount of compound employed in the practice of the present invention will depend upon the type of response desired, the formulation used and the type of plant treated as shown in the following examples.

Alternatively, and according to another embodiment, the present invention provides for a method for restoring normal anther development in the genetically modified male sterile plant of the invention by applying on male sterile flowers of the plant a composition containing an inhibitor of the 11-/12-OHJA sulfotransferase and an acceptable carrier.

### EXAMPLES

The following examples are illustrative of the wide range of applicability of the present invention. The invention is not restricted to the production of male sterile tobacco plants but can be applied to all flowering plant species. It should readily occur that the recognition of producing male sterile plants according to methods of the present invention in connection with other plants not specifically illustrated herein, is readily within the capabilities of one skilled in the art. The following examples are intended only to illustrate the invention and are not intended to limit its scope. Modifications and variations can be made therein without departing from the spirit and scope of the invention.

The following experimental procedures and materials were used for the examples set fort below.

A) Material and Methods

Studies using a vector:

For transgenic studies an EcoRI-HindIII cassette, from the plasmid pBI-525 comprising two CaMV 35S promoters in tandem followed by an AMV translational enhancer and a NOS terminator, was ligated to the plasmid pBI-101 which was previously digested with the same restriction endonucleases. The resulting vector called pBI-101-525 contained two CaMV 35S minimal promoters in tandem followed by an AMV translational enhancer, a NOS terminator and a kanamycin resistance gene. AtST2a cDNA (SEQ ID NO 1; FIG. 7) was cloned in the sense orientation at the BaMHI site in a polylinker lying downstream of the AMV enhancer. Various other promoters may be used to drive the expression of an exogenous gene in a plant. For example the ubiquitin promoter may be used for constitutive expression. Alternatively, inducible promoters may also be used such as the ethanol-inducible promoter or the glucocorticoid-inducible promoter.

Agrobacterium Transformation:

A. tumefaciens strain LBA4404 was transformed with the AtST2a-pBI-101-525 sense construct by the method described in Gynheung et al. (1988) Biology Manual, A3:1-19.

Nicotiana tabacum Transformation:

Transgenic tobacco plants were produced using the leaf disk transformation method described by Horsch, R. B. et al. (1984) Science, Vol. 227,1229-1231.

Northern Blot of mRNA Extracts

Total RNA was extracted from frozen tissues by the use of phenol/chloroform/isoamyl alcohol 25:24:1 according to the method described by Sambrook et al (1989). 20 μg of total RNA per lane was subjected to electrophoresis and Northern blot analysis was performed according to Sambrook et al. (1989). Blots were hybridized at 65° C. for 16 h with a ³²P-labelled fragment of A. thaliana AtST2a cDNA encompassing the entire coding sequence.

Western Blot Analysis

For the analysis of AtST2a expression, leaves from T1 and T2 plants were ground in liquid nitrogen, and the powder was boiled in 2×SDS sample buffer. Protein extracts were separated by SDS-polyacrylamide gel electrophoresis on 12% polyacrylamide gels and transferred onto nitrocellulose membrane. AtST2a was immunodetected using anti-AtST2a polyclonal antibodies (dilution 1:1000) and goat anti-rabbit secondary antibodies conjugated with alkaline phosphatase (dilution 1:3000; Bio Rad™). To confirm equal loading of each sample, protein extracts were run on SDS-PAGE and stained with Coomassie blue.

Plant Growth Conditions

Wild type (SNN) and transgenic plants were grown in soil under greenhouse conditions with a 16 hours photoperiod.

Normalization Experiments

The apex of T2 plants of line 7 was soaked daily in a solution containing either: 0.05% Tween20 in water (control), 0.05% Tween20 in water containing 50 μM MeJA or 0.05% Tween20 in water containing 100 μM 12-OHJA. The treatments were started approximately 7 days before the appearance of the first flower buds and lasted a total of 14 days. A minimum of 5 plants for each treatment were analyzed in this study.

B) Results

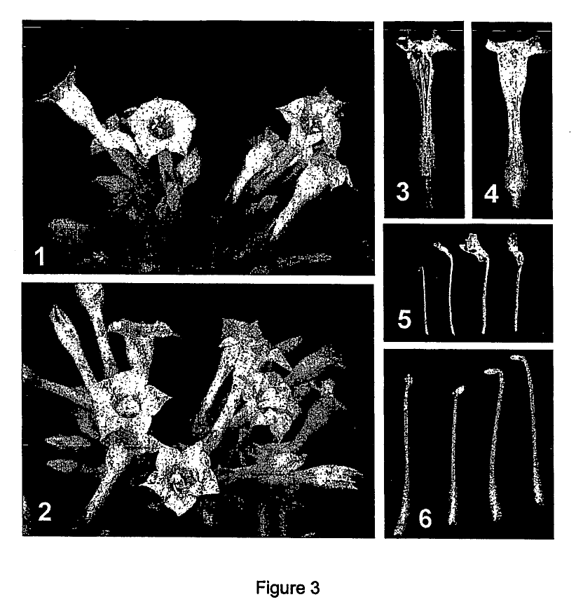

The inventors demonstrated that it is possible to generate male sterile transgenic tobacco plants by heterologous expression of the A. thaliana gene AtST2a encoding the 12-OHJA sulfotransferase. The AtST2a gene was introduced in Nicotiana tabacum plants by Agrobacterium-mediated transformation. Plants were regenerated and transformed plants were selected by resistance to kanamycin. Transformation was confirmed by Southern, Northern and Western blot. 29 independent transgenic lines were generated. FIG. 2 shows a photograph of a Northern blot for three selected transgenic lines. Plants from four lines (including line 7) exhibited a male sterile phenotype and the phenotype was found to correlate with a high level of AtST2a expression (FIG. 3 and 2C). Except for the male sterile phenotype, no other phenotypic alterations were observed in the transgenic lines.

These results clearly indicate that it is possible to produce male sterile plants by altering the level of the enzyme that sulfonates 12-OHJA. It is also predicted that inhibition of the expression of the endogenous jasmonic acid 11-/12-hydroxylase will produce the same effect.

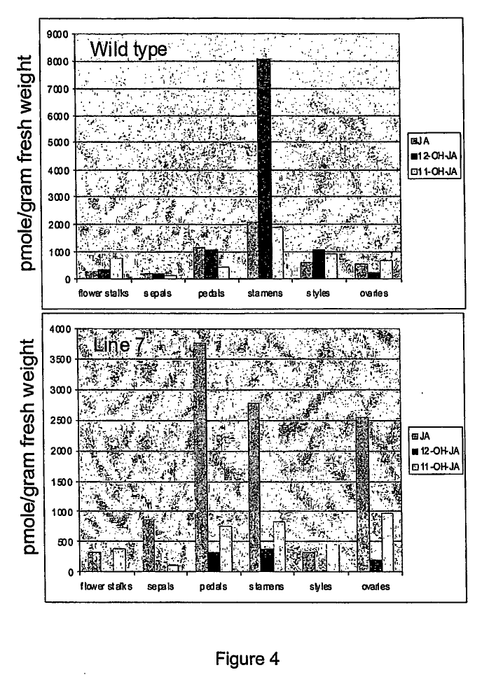

The levels of different jasmonates were quantified in dissected flower tissues of the transgenic line 7 as compared with wild type. The results presented in FIG. 4 show that anthers are the preferential sites of accumulation of 12-OHJA in wild type flowers. In contrast, a drastic reduction in the amount of 12-OHJA in the anthers of the transgenic plant was observed as compared with wild type. These results confirm that the overexpression of the 12-OHJA sulfotransferase lead to a decrease of 12-OHJA in the anthers of transgenic tobacco plants.

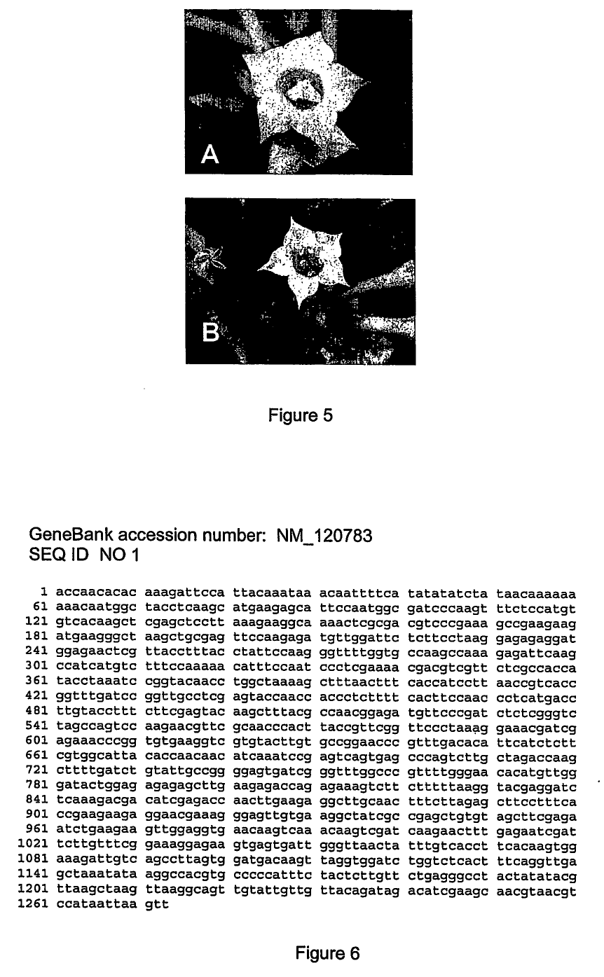

In order to restore the phenotype, the apex of transgenic plants (line 7) were soaked daily for 2 minutes in a solution containing 100 μM 12-OHJA dissolved in aqueous 0.05% TWEEN20. The application was started approximately 7 days prior to the appearance of the first flower bud and was continued for 7 days after the first flower appeared. The results show that the application of 12-OHJA restored normal anther development (FIG. 5). Similar results were obtained with 50 μM MeJA which is a precursor of 12-OHJA biosynthesis (data not shown). Control treatments with the carrier solution did not restore normal anther development. Application of 12-OHJA was also shown to restore the male sterile phenotype of the A. thaliana opr3 mutant (data not shown).

These results demonstrate that the male sterile phenotype associated with a deficiency in 12-OHJA observed in the tobacco transgenic line 7 and in the A. thaliana opr3 mutant can be restored by the application of 12-OHJA.

1. A method for producing a male sterile plant characterized in that said method comprises the step of decreasing the level of 11-and/or 12-hydroxyjasmonate by increasing in said plant the level of in-vivo sulfonation of hydroxyjasmonates or decreasing the level of synthesis of 11-and/or 12-hydroxyjasmonate.

2. The method of claim 1, characterized in that the level of in-vivo sulfonation of hydroxyjasmonates is increased by increasing in said plant the endogenous activity of a hydroxyjasmonate sulfotransferase.

3. The method of claim 1, characterized in that the level of synthesis of 11- and/or 12-hydroxyjasmonate is decreased by decreasing in said plant the activity of a jasmonic acid 11-/12-hydroxylase.

4. The method according to any one of claims 1 to 3, characterized in that the increasing of the level of in-vivo sulfonation of hydroxyjasmonates or the decreasing of the level of synthesis of 11-and/or 12-hydroxyjasmonate is achieved by a process selected from the group consisting of genetic modification of said plant, radiation mutagenesis of said plant, chemical mutagenesis of said plant and selection of natural mutants.

5. The method of claim 4, characterized in that said method consists of genetic modification.

6. The method of claim 5, characterized in that the endogenous activity of the sulfotransferase is increased by stimulating the expression of at least one gene selected from the group consisting of: AtST2a, AtST2b, functional homologues thereof having at least 50% homology with SEQ ID no. 1 or SEQ ID no. 2 and a nucleic acid encoding for an amino acid sequence having at least 50% homology with amino acid sequence of SEQ ID no. 3 or SEQ ID no. 4.

7. A plant cell transformation vector capable of facilitating transfer and expression of an exogenous nucleic acid into an isolated cell and/or facilitating integration of an exogenous nucleic acid into genome of said cell, characterized in that said vector comprises at least one promoter sequence, one enhancer sequence and one exogenous nucleic acid sequence, said promoter being a constitutive expression promoter or an inducible promoter, said exogenous nucleic acid being selected from the group consisting of nucleic acid sequence having at least 50% homology with SEQ ID no. 1 or SEQ ID no. 2 and a nucleic acid encoding for an amino acid sequence having at least 50% homology with amino acid sequence of SEQ ID no. 3 or SEQ ID no. 4.

8. The vector of claim 7, characterized in that the inducible promoter is an ethanol-inducible promoter or a glucocorticoid-inducible promoter.

9. The vector of claim 7, characterized in that the constitutive expression promoter is an ubiquitin promoter.

10. The vector of claim 7, characterized in that the promoter is CaMV 35S promoter.

11. The vector of claim 7, characterized in that the enhancer is an AMV translational enhancer.

12. A method for producing a male sterile plant, characterized in that said method comprises:
introducing into a cell of a suitable plant an exogenous nucleic acid molecule via the vector as defined in any one of claims 7 to 11;
regenerating a transgenic plant from said cell; and where necessary
growing the transgenic plant for a time and under conditions sufficient to permit expression of the exogenous nucleic acid sequence and thereby stimulating expression of the hydroxyjasmonic acid sulfotransferase.

13. A genetically modified male sterile plant, characterized in that the endogenous level of 11- or 12-hydroxyjasmonate in said genetically modified male sterile plant is lower than the endogenous level of 11- or 12-hydroxyjasmonate in a non genetically modified plant.

14. A genetically modified male sterile plant, characterized in that the endogenous level of 11- or 12-hydroxyjasmonic acid sulfotransferase in said genetically modified male sterile plant is higher than the endogenous level of 11- or 12-hydroxyjasmonic acid sulfotransferase in a non genetically modified plant.

15. A composition for restoring normal anther development in a genetically modified male sterile plant, comprising at least one jasmonate and an acceptable carrier.

16. The composition of claim 15, characterized in that the jasmonate is selected from the group consisting of: 11-hydroxyjasmonic acid, 12-hydroxyjasmonic acid, glucoside of 11-hydroxyjasmonic acid, glucoside of 12-hydroxyjasmonic acid, 11-hydroxymethyljasmonic acid, glucoside of 11-hydroxymethyljasmonic acid, 12-hydroxymethyljasmonic acid and glucoside of 12-hydroxymethyljasmonic acid.

17. The composition of claim 15, characterized in that the composition contains 100 μM of 12-hydroxyjasmonic acid or 50 μM methyl-jasmonic acid.

18. The composition of claim 17, characterized in that the composition further contains TWEEN20™.

19. The composition of claim 18, characterized in that the composition contains TWEEN20™ in the concentration of 0.05 weight percent of the total volume of said composition.

20. A method for restoring normal anther development in a genetically modified male sterile plant as defined in any one of claims 13 or 14, comprising a step of applying on male sterile flowers of said plant a composition as defined in any one of claims 15 to 19.

21. The method of claim 20, characterized in that the step of applying said composition on male sterile flowers of said plant is achieved by soaking male sterile flowers in said composition for two minutes daily starting 7 days before appearance of a first flower bud and ending 7 days after appearance of a first flower.

22. A composition for restoring normal anther development in a genetically modified male sterile plant, comprising at least one 11-/12-OHJA sulfotransferase inhibitor and an acceptable carrier.

23. A method for restoring normal anther development in a genetically modified male sterile plant as defined in any one of claims 13 or 14, comprising a step of applying on male sterile flowers of said plant a composition as defined in claim 22.
